(19) United States
(12) Patent Application Publication
(10) Pub. No.: US 2026/0185037 A1
(43) Pub. Date: Jul. 2, 2026

(54) High Protein Producing Yeast Strains and Uses Thereof

(71) Applicant: NextFerm Technologies Ltd., Yokneam lllit (IL)

(72) Inventors: Yaniv HARARI, Matsli'ah (IL); Shaked AMSALEM, Haifa (IL); Ira LAVIN, Kiryat Yam (IL); Gai BEN-DROR, Beer Sheva (IL)

(21) Appl. No.: 19/130,628

(22) PCT Filed: Nov. 17, 2023
(86) PCT No.: PCT/IB2023/061632

§ 371 Date: May 16, 2025

(30) Foreign Application Priority Data
Nov. 17, 2022 (EP) 22208130.9

(51) Int. Cl. C12N 1/185; C07K 14/395; C12R 1/865

(52) U.S. Cl. CPC C12N 1/185; C07K 14/395; C12R 2001/865

(57) ABSTRACT

The invention relates to Saccharomyces cerevisiae strains exhibiting high growth rates and producing high amounts of protein, uses thereof for the production of yeast protein isolate and yeast protein isolate obtained by culturing of the high protein producing yeast strains. The invention moreover provides yeast protein isolate and methods for producing the same, taking advantage of the advantageous properties of the Saccharomyces cerevisiae strains disclosed herein, as well as uses of the high protein producing strains of S. cerevisiae.

### FIELD OF THE INVENTION

The invention relates to Saccharomyces cerevisiae strains exhibiting high growth rates and producing high amounts of protein, uses thereof for the production of yeast protein isolate and yeast protein isolate obtained by culturing of the high protein producing yeast strains. The invention moreover relates to yeast protein isolate and methods for producing the same, taking advantage of the advantageous properties of the Saccharomyces cerevisiae strains disclosed herein, as well as uses of the high protein producing strains of S. cerevisiae.

### BACKGROUND OF THE INVENTION

Protein extracts or isolates for nutritional purposes are commonly obtained from various animal, plant or microbial sources, including bacteria and yeast. One of the advantages of using yeast as a source of protein for human or animal consumption is that yeast protein comprises all amino acids essential for humans. Yeast can moreover be cultured on an industrial scale using well established methods.

Baker's yeast (Saccharomyces cerevisiae) is used extensively for the bakery industry and for the production of yeast extract, due to its good fermentative capacity, storage stability and ease of handling. However, S. cerevisiae is a Crabtree-positive yeast species, which means that strains of S. cerevisiae commonly produce ethanol under aerobic conditions at high concentrations of fermentable carbon source (e.g., glucose, sucrose and the like), which are required for fast growth, i.e., fast accumulation of biomass. The production of ethanol is at the expense of biomass accumulation and therefore undesired in industrial yeast protein production. The tolerance to high carbon source concentrations without ethanol formation is specific for particular strains of yeast, or families of strains that are genetically closely related. Accordingly, there is a need for highly productive yeast strains that can tolerate high carbon source feed rates without switching to an anaerobic metabolic state.

Industrial strains of S. cerevisiae used for yeast extract production are commonly selected for their capability of producing high amounts of protein, growth rate and/or accumulation of biomass during fermentation. Higher protein yields per amount of biomass and per volume of fermentation culture will also enable the more economic production of yeast protein isolate. Ideal strains for large-scale production of yeast protein isolate produce high amounts of protein, while exhibiting high growth rates to enable the fast accumulation of biomass during fermentation.

The invention provides strains of S. cerevisiae with highly attractive properties for industrial production of yeast protein and yeast extract, including a short fermentation cycle, the capability of quickly accumulating high biomass during fermentation, including fermentation at high rates of carbon source feed which leads to high growth rates, results in a biomass with very high protein content, and high protein purity and recovery during downstream processing.

### SUMMARY OF THE INVENTION

In one aspect, the invention relates to a strain of Saccharomyces cerevisiae, obtainable from strain NFY-1816, deposited as CBS148506.

In one embodiment, the strain is obtainable by breeding between a first strain and a second strain, wherein the first and/or the second strain is strain NFY-1816, deposited as CBS148506.

In a further aspect, the invention relates to a strain of S. cerevisiae, wherein the strain is NFY-1816, deposited as CBS148506.

In one embodiment of the abovementioned strains, the strain comprises a protein content of at least about 50% (w/w), relative to the dry yeast biomass.

In a further embodiment, the strain is capable of producing yeast protein in an amount of at least about 50 g/L of fermentation culture volume.

In a further embodiment, the strain enables a growth rate (u) of at least about 0.29.

In yet another aspect, the invention relates to a method for producing yeast protein isolate, comprising culturing a strain of S. cerevisiae described herein.

In one embodiment, the method further comprises disruption of yeast cells to obtain a lysate, removal of cell wall components to obtain a cleared lysate and isolation of the yeast protein from the lysate.

In another aspect, the invention relates to a yeast protein isolate obtainable by the method described herein.

In another aspect, the invention relates to a yeast protein isolate produced by the method described herein.

In one embodiment, the yeast protein isolate comprises a protein content of at least about 70% (w/w).

In yet a further aspect, the invention relates to a use of a strain of S. cerevisiae as described herein for the production of yeast protein isolate.

### DETAILED DESCRIPTION OF THE INVENTION

The inventors have, through random mutagenesis and careful selection for desired properties, obtained strains of S. cerevisiae with highly attractive properties for the industrial production of yeast protein.

In one aspect, the invention relates to a strain of Saccharomyces cerevisiae, obtainable from strain NFY-1816. NFY-1816 has been deposited as CBS148506 (WESTERDIJK FUNGAL BIODIVERSITY INSTITUTE, CBS, Uppsalalaan 8, P.O. Box 85167, 3508 AD Utrecht, NL) on Nov. 4, 2021. In one embodiment, the strain is obtainable by breeding between a first strain and a second strain, wherein the first or the second strain is strain NFY-1816, deposited as CBS148506. In another embodiment, the strain is obtainable by breeding between a first strain and a second strain, wherein the first and the second strain is strain NFY-1816, deposited as CBS148506.

Breeding between individual strains of S. cerevisiae occurs by mating, i.e., the fusion of haploid yeast cells of opposite mating types (mating type A or alpha).

In a further aspect, the invention provides a strain of S. cerevisiae, wherein the strain is NFY-1816, deposited as CBS148506.

The strains according to the invention are characterized by their exceptionally high production of yeast protein, relative to their biomass. Thus, in one embodiment, the strains described herein comprise a protein content of at least about 50% (w/w), at least about 55% (w/w), at least about 60% (w/w), at least about 65% (w/w), at least about 70% (w/w) or at least about 74% (w/w) relative to the dry yeast biomass. Preferably, the strains described herein comprise a protein content of at least about 60% (w/w) relative to the dry yeast biomass. More preferably, the strains described herein comprise a protein content of at least about 65% (w/w) relative to the dry yeast biomass. Even more preferably, the strains described herein comprise a protein content of at least about 70% (w/w) relative to the dry yeast biomass. Most preferably, the strains described herein comprise a protein content of at least about 74% (w/w) relative to the dry yeast biomass.

As used herein, the term “v/v” means “volume/volume” and “w/w” means “weight/weight”.

As used herein, the term “about” in reference to a value encompasses not only that precise value but also values±5% of that value.

The protein content is commonly determined after fermentation under suitable conditions, separating of the fermented yeast cells from the growth medium to remove or essentially remove residual medium and optionally subsequent drying (e.g., by lyophilization). Suitable fermentation conditions are described hereinbelow.

The “protein content” of yeast is usually determined by measuring the amount of nitrogen in a given sample by various methods know to the skilled artisan (e.g., M. Hayes, Foods 2020, 9, 1340). In a preferred embodiment, the protein content is determined as follows. The total nitrogen content is determined using the Kjeldahl method (Christensen and Fulmer, Plant. Physiol. 1927, 2 (4), 455-460) and the result multiplied with 6.25. In a further preferred embodiment, the protein content is determined using the method according to Dumas. Total nitrogen content is determined according to Dumas and the result multiplied with 6.25.

The provided strains possess an unusually high protein productivity relative to the fermentation volume, which renders them particularly well suited for industrial scale yeast protein production. Accordingly, the strains according to the invention are capable of producing yeast protein in an amount of at least about 50 g/L, at least about 55 g/L, at least about 60 g/L or at least about 65 g/L of fermentation culture volume.

A further advantageous property of the yeast strains disclosed herein lies in their high growth rate without switching from aerobic fermentation to anaerobic respiration. Aerobic fermentation is the metabolic process by which yeast cells metabolize carbon sources (sugars) via fermentation in the presence of oxygen and occurs through the repression of normal respiratory metabolism. Aerobic fermentation allows proliferating cells to convert nutrients such as glucose and glutamine more efficiently into biomass, as compared to anaerobic respiration, by avoiding unnecessary catabolic oxidation of such nutrients into carbon dioxide. Some yeast species (including S. cerevisiae) switch to anaerobic respiration at high external glucose concentrations rather than producing biomass via the tricarboxylic acid (TCA) cycle, the usual process occurring aerobically in most yeasts. This phenomenon is referred to as the Crabtree effect. Increasing the carbon sources in the fermentation broth results in an increase of the growth rate of the cultured yeast cells, up to the point where the cells switch from aerobic fermentation to anaerobic respiration, which sets a limit for the maximum growth rate that may be achieved. The highest achievable growth rate is specific for the species and strain of yeast that is used.

The yeast strains disclosed herein have been observed to enable growth rates (u) of at least about 0.29, at least about 0.30, at least about 0.31 or at least about 0.32. Preferably, the yeast strains enable a growth rate (μ) of at least about 0.30, more preferably at least about 0.31 and most preferably at least about 0.32. In further specific embodiments, the yeast strains enable a growth rate (μ) of about 0.29, about 0.30, about 0.31 or about 0.32. Preferably, the yeast strains enable a growth rate (μ) of about 0.30, more preferably about 0.31 and most preferably about 0.32.

As used herein, the growth rate “μ” refers to the hourly growth rate calculated according to the equation in (biomass at T2/biomass at T1)/(T2−T1), wherein “T1” is a specific time point and “T2” is a specific time point later than “T1”.

The yeast strain of the invention may be capable of fermenting glucose and sucrose. The yeast strain may be capable of assimilating carbon from glucose and sucrose, as well as, optionally, maltose, galactose and/or raffinose. Typically, melibiose, and optionally, galactose and/or raffinose, cannot be assimilated. The strains of the invention each demonstrate a typical sugar spectrum to assimilate and grow on it.

In a further aspect, the invention provides a method for producing yeast protein isolate, comprising culturing a strain of S. cerevisiae disclosed herein.

In a specific embodiment, the method comprises disruption of yeast cells to obtain a lysate, removal of cell wall components to obtain a cleared lysate and isolation of the yeast protein from the lysate. In a further embodiment, the method comprises separation and isolation of the soluble fraction after isolation of the yeast protein from the lysate. The soluble fraction may further be dried to obtain a soluble fraction powder. In yet another embodiment, the method comprises removal of cell wall components and isolation of the cell wall component fraction.

A “yeast protein isolate” (YPI) according to the present application is the protein content of a yeast cell, or a plurality of yeast cells, that has largely been cleared of impurities.

The high protein productivity of the yeast strains provided herein results in a high degree of purity of the yeast protein isolate obtained by fermentation of the yeast strains. More specifically, the yeast strains were observed to produce yeast protein isolate with a protein content of at least about 70% (w/w), at least about 75% (w/w), at least about 80% (w/w) or at least about 83% (w/w), relative to the mass of the YPI. Preferably, the yeast strains produce yeast protein isolate with a protein content of at least about at least about 75% (w/w), more preferably at least about 80% (w/w) and most preferably at least about at least about 83% (w/w), relative to the mass of the YPI. The high degree of purity of the YPI obtained from the strains of the invention advantageously necessitates less rigorous downstream purification of the YPI. In addition, a high protein content YPI product, may extend the scope of potential applications, by addressing to products with high protein content demand.

The high protein productivity of the strains disclosed herein moreover results in an exceptionally high extractability and recovery of yeast protein. The extractability and recovery of yeast protein from isolated yeast cells (i.e., yeast biomass) may be determined by subjecting the yeast biomass to partial cell disruption, removal of the non-soluble fraction, optionally further extraction of protein from the non-soluble fraction and subsequent determination of the protein content of the isolated soluble fraction(s). The protein content determined this way is subsequently compared to the amount of protein extracted from a comparator strain that has been treated in the same manner. The extractability or extraction yield referred to herein is a relative measure that requires comparison to an alternative yeast strain, e.g., a commercially available yeast strain for YPI production.

The extractability and recovery of yeast protein may be determined as described in the examples (cf. in particular example 4). For example, extractability may be determined as follows. A defined amount of yeast biomass (e.g., compressed yeast biomass) of a first yeast strain is dispersed in cold water, the resulting suspension subjected to cell disruption by passing through a high-pressure continuous homogenizer (e.g., one pass at 900-1,000 bar, which leads to partial cell disruption) with continued cooling at the outlet of the homogenizer. A sample of the obtained homogenate is taken and the protein content is determined. The non-soluble fraction containing cell walls and non-disrupted cells is removed (e.g., by centrifugation at 4,000 RPM, 4° C.). The resulting pellet is washed with water, separated again (e.g., by centrifugation at 4,000 RPM, 4° C.) and the total amount of protein in the combined supernatants is determined and compared to the initial protein content of the sample to obtain an extractability measure. The same procedure is repeated with a second (comparator) yeast strain and the extraction yields of the first and the second yeast strain are compared.

Without wishing to be bound by theory, it may be assumed that the yeast strains according to the invention exhibit favourable extractability due to a combination of high amounts of protein with an advantageous chemical composition and physical characteristics of the yeast cell walls.

The soluble fraction obtained after isolation of the yeast protein from the lysate according to the method for producing yeast protein isolate disclosed herein is generally largely cleared from yeast protein, but may comprise varying amounts of compounds of commercial value, including amino acids (e.g., glutamic acid) and nucleic acids (e.g., ribonucleic acid). The inventors have observed that the soluble fraction obtained from the yeast strains described herein comprise unusually high levels of glutamic acid (on a salt-free basis) and nucleic acids (on a salt-free basis). For example, the soluble fraction may comprise at least about 6% of glutamic acid (on a salt-free basis) and at least about 20% nucleic acids (on a salt-free basis) relative to the dry mass of the soluble fraction. In a specific embodiment, the soluble fraction comprises at least about 8% of glutamic acid (on a salt-free basis) and at least about 25% nucleic acids (on a salt-free basis) relative to the dry mass of the soluble fraction. Preferably, the soluble fraction comprises at least about 10% of glutamic acid (on a salt-free basis) and at least about 30% nucleic acids (on a salt-free basis) relative to the dry mass of the soluble fraction.

Glutamic acid content can be determined according to established analytical methods, for example by derivatization with a chromophore (e.g., 6-aminoquinolyl-N-hydroxysuccinimidyl carbamate, AQC) and high-performance liquid chromatography (HPLC) (Cohen et al., Anal. Biochem. 1993, 211 (2), 279-287). Methods for the quantification of nucleic acids are well known in the art.

The cell wall component fraction obtained after removal of cell wall components according to the method for producing yeast protein isolate disclosed herein comprises varying degrees of beta-glucans. The yeast strains disclosed herein have been observed to provide a cell wall component fraction with an unusually high content of beta glucans. More specifically, the cell wall component fraction may comprise at least about 25% (w/w), preferably, at least about 30% (w/w) and more preferably at least about 35% (w/w) beta glucans relative to the dry cell wall component fraction.

In a further aspect, the invention relates to a yeast protein isolate (YPI) obtainable by the method for producing yeast protein isolate disclosed herein.

In another aspect, the invention provides a yeast protein isolate produced by the method for producing yeast protein isolate disclosed herein.

In yet another aspect, the invention relates to the use of the high protein producing strains of S. cerevisiae disclosed herein for the production of yeast protein isolate.

The high protein producing strains of S. cerevisiae disclosed herein may further be used as as a dietary supplement, such as a nutritional supplement, a food supplement, a beverage supplement, and for food additives. For example, the strains may be incorporated into the diet (e.g., feed) of farm-grown animals. Thus, in one aspect, the invention relates to the use of the high protein producing strains of S. cerevisiae disclosed herein for the production of a product comprising YPI, wherein the product is a dietary supplement, a nutritional supplement, a food supplement, a beverage supplement, a food additive or a feed additive.

The invention further provides a use of the soluble fraction obtained after isolation of the yeast protein from the lysate according to the method for producing yeast protein isolate disclosed herein as a food additive, e.g., as flavoring.

Accordingly, the method for producing yeast protein isolate disclosed herein may yield a yeast protein isolate with a protein content or result in an extraction yield, a soluble fraction an/or a cell wall component fraction as disclosed hereinabove, including all embodiments.

### EXAMPLES

### Example 1: Strain NFY-1816 Yields High Protein Levels

Fermentation of strain NFY-1816 was conducted in a 5-liter laboratory fermenter. The initial fermentation broth, before starter inoculation, was composed of: corn steep liquor, nitrogen sources (ammonium sulfate and corn steep liquor), mono-potassium phosphate vitamins (biotin, calcium pantothenate, nicotinic acid, inositol, thiamine hydrochloride, pyridoxine hydrochloride and para-aminobenzoic acid) and minerals (zinc sulfate, manganese sulfate, copper sulfate, cobalt sulfate, sodium molybdate, calcium chloride, ferrous sulfate and magnesium sulfate).

Growth was controlled by continued addition of 50% glucose solution and 10% aqueous ammonia solution, agitation at 500-1000 RPM, air feed at 6 L/min to maintain oxygen saturation between 60-100%. Cooling was applied to control temperature between 29-31° C. and ammonia and sulfuric acid were fed in order to maintain pH between 4.5 and 6.5.

After 14.1 h of fermentation, when the volume of the fermentation broth was 3 L and yeast dry mass concentration was 69.1 g/L, the total volume of the fermenter was harvested by vacuum Buckner filtration. The filtrate was removed, while the solids were washed two times with 3 L of water to obtain the final yeast biomass. A sample of the obtained yeast biomass was lyophilized for 48 h and the dried sample was subjected to protein content analysis to provide a result of 74.0% (w/w) protein content.

### Example 2: Growth Rate of Strain NFY-1816

A fermentation reaction equivalent to the one described in Example 1 was performed with a specifically high glucose feed rate of up to 0.55 g glucose (100% basis)/g yeast/hr.

Between hour 4 and hour 5 the growth rate was determined to be μ=0.324. No switch into anerobic respiration was observed.

The fermentation was terminated after 14.9 h resulting in a biomass of 75.5 g/L.

### Example 3: Protein Productivity of Strain NFY-1816

A molasses-based fermentation of strain NFY-1816 was conducted in a 5 L lab fermenter.

The initial fermentation broth, before starter inoculation, was composed of: beet-molasses, nitrogen sources (ammonium sulfate, di-ammonium phosphate and corn steep liquor), vitamins (biotin, calcium pantothenate, nicotinic acid, inositol, thiamine hydrochloride, pyridoxine hydrochloride and para-aminobenzoic acid) and minerals (zinc sulfate, manganese sulfate, copper sulfate, cobalt sulfate, sodium molybdate, calcium chloride, ferrous sulfate and magnesium sulfate).

Growth was controlled by continued addition of molasses (40 degrees Brix) solution and 10% aqueous ammonia solution, agitation at 500-1000 RPM, air feed at 6 L/min to maintain oxygen saturation between 60-100%. Cooling was applied to control temperature between 29-31° C. and ammonia and sulfuric acid were fed in order to maintain pH between 4.5 and 6.5.

The fermentation was performed with maximizing molasses feed and fermentation time to obtain after 17 h, 94.5 g/L biomass with a protein content of 70.5%, to a total of 66 g/L protein per volume of fermentation broth.

### Example 4: Comparative Assessment of Protein Extractability

300 g of compressed yeast biomass (with 20%-30% dry matter) obtained from fermentation according to the procedure described in Example 3 was dispersed in 300 g of cold water and was subjected to cell disruption by one pass through high pressure continuous homogenizer (at 900-1,000 bar) with continued cooling at the outlet of the homogenizer.

A sample of the obtained homogenate was taken, the protein content was determined, the sample was spun down in a laboratory centrifuge (20 min, 4,000 RPM, 4° C.) to remove the non-soluble fraction containing cell walls and non-disrupted cells. Water was added to the pellet to reach the same volume of the original homogenate, the pellet was washed and separated again by centrifuge in the same conditions. The supernatant obtained was combined with the first supernatant and the total amount of protein in the combined supernatants was determined and compared to the initial protein content of the sample (which was determined by the measurement of the homogenate sample). The partial extraction yield was determined as 46.5%.

Same procedure was performed on a commercially available yeast strain (Commercial Yeast 1), resulting in a partial extraction yield of 26.5%.

### Example 5: Comparative Analysis of YPI Obtained from Strain NFY-1816

A sample of biomass obtained from fermentation of strain NFY-1816 according to the procedure described hereinabove was dispersed in cold water (to final dry mass concentration of about 5%) and subjected to 3 passes in a continuous high-pressure homogenizer at 900-1,000 bar, the homogenizer outlet was cooled continuously.

The obtained homogenate was subjected to centrifugal separation in lab centrifuge (20 min, 4,000 RPM, 4° C.) and the soluble fraction (lysate) was separated from the pellet and collected as supernatant. The pellet was washed again with additional amount of cold water (about 50% of the original amount of water), mixture was separated by centrifuge in same conditions as above, the supernatant was collected and united with the first supernatant.

The pH of the lysate was increased to 10 with NaOH (20%). The basic lysate was heated to 70° C. in shaker incubator and was held at those conditions for 5-7 h, allowed to cool to room temperature and subjected to pH adjustment to 5.8 with HCl (3.5%).

The obtained pH adjusted slurry was subjected to centrifugal separation (10 min., 4,000 RPM, 20° C.).

The supernatant was subjected to concentration by evaporation under vacuum until 30% solid content level was obtained and the concentrated material was centrifuged again and the clear liquid (supernatant) was dried in lab spray dryer to obtain the soluble fraction powder.

The solids obtained from the centrifugation of the pH adjusted slurry were washed 3 times and dried by lyophilization for 48 hours to obtain the (purified) YPI.

Both YPI and soluble fraction powder were analyzed for composition.

Yield from biomass refers to the yield of YPI per dry yeast biomass. Protein content refers to protein content (w/w) of the isolated YPI (dry mass) and was determined as described hereinabove. Taste: Samples assessed by panel of experts in protein tasting-average score (1 to 4 scale with 1=worst and 4=best).

Glutamic acid and nucleic acids content is indicated in percent (w/w) per dry mass of the soluble fraction.

The same procedure was performed with commercially available yeast strain biomasses (commercial yeast 1 to 3). Results are summarized in the following tables.

YPI

| | Yield from | Protein | Nucleic acids |
|---|---|---|---|
| Yeast source | biomass | content | content |
| NFY-1816 | 59.6% | 83.4% | 1.35% |
| Commercial yeast 1 | 50.6% | 79.8% | 1% |
| Commercial yeast 2 | 42.7% | 78.7% | 0.62% |
| Commercial yeast 3 | 49.4% | 80.4% | 0.64% |

Soluble fraction powder

| | Glutamic acid | Nucleic acids | |
|---|---|---|---|
| Yeast source | (salt free basis) | (salt free basis) | Taste |
| NFY-1816 | 11% | 31% | 4 |
| Commercial yeast 1 | 4.1% | 25% | 2 |
| Commercial yeast 2 | 2% | 18% | 1 |
| Commercial yeast 3 | 4.5% | 27% | 2 |

### Example 6: Analysis of Composition of Cell Wall Component Fraction

1000 g of sample of biomass obtained from fermentation of strain NFY-1816_according to the procedure as described hereinabove was dispersed in 1000 g of cold water and subjected to 3 passes in continuous high-pressure homogenizer at 900-1,000 bar, the homogenizer outlet was cooled continuously.

The obtained homogenate was subjected to centrifugal separation in lab centrifuge (20 min., 4,000 RPM, 4° C.) and the soluble fraction (lysate) was collected as supernatant. The solid phase, predominantly composed of the cell walls, was washed twice and reseparated. The pellet was dispersed in water, heat treated (85° C. 5 min.), spray dried and submitted to analysis of beta glucans. Results showed 37.7% beta glucans out of total dried cell wall component fraction. The same procedure was performed with a commercially available yeast product in the market (Commercial yeast 1) and results showed 24.6% beta glucans out of the total dried cell wall component fraction.

### ITEMS OF THE INVENTION

1. A strain of Saccharomyces cerevisiae, obtainable from strain NFY-1816, deposited as CBS148506.
2. S. cerevisiae strain according to item 1, wherein the strain is obtainable by breeding between a first strain and a second strain, wherein the first and/or the second strain is strain NFY-1816, deposited as CBS148506.
3. A strain of S. cerevisiae, wherein the strain is NFY-1816, deposited as CBS148506.
4. Strain according to any one of items 1 to 3, comprising a protein content of at least about 50% (w/w), relative to the dry yeast biomass.
5. Strain according to item 4, comprising a protein content of at least about 55% (w/w), at least about 60% (w/w), at least about 65% (w/w), at least about 70% (w/w) or at least about 74% (w/w) relative to the dry yeast biomass.
6. Strain according to any one of items 4 or 5, wherein the protein content is determined by
   a) determining the total nitrogen content using the Kjeldahl method and multiplying the result with 6.25, or
   b) determining the total nitrogen content according to Dumas and multiplying the result with 6.25.
7. Strain according to any one of items 1 to 6, capable of producing yeast protein in an amount of at least about 50 g/L of fermentation culture volume.
8. Strain according to item 7, capable of producing yeast protein in an amount of at least about 55 g/L, at least about 60 g/L or at least about 65 g/L of fermentation culture volume.
9. Strain according to any one of items 1 to 8, wherein the strain enables a growth rate (μ) of at least about 0.29.
10. Strain according to item 9, wherein the strain enables a growth rate (μ) of at least about 0.30, at least about 0.31 or at least about 0.32.
11. A method for producing yeast protein isolate, comprising culturing a strain of S. cerevisiae according to any one of items 1 to 10.
12. Method according to item 11, further comprising disruption of yeast cells to obtain a lysate, removal of cell wall components to obtain a cleared lysate and isolation of the yeast protein from the lysate.
13. Method according to any one of items 11 or 12, further comprising separation and isolation of the soluble fraction after isolation of the yeast protein from the lysate and optionally drying of the soluble fraction to obtain a soluble fraction powder.
14. Method according to any one of items 11 to 13, further comprising removal of cell wall components and isolation of the cell wall component fraction.
15. Yeast protein isolate obtainable by the method according to any one of items 11 to 14.
16. Yeast protein isolate produced by the method according to any one of items 11 to 14.
17. Yeast protein isolate according to any one of items 15 or 16, comprising a protein content of at least about 70% (w/w).
18. Yeast protein isolate according to item 17, comprising a protein content of at least about 75% (w/w), at least about 80% (w/w) or at least about 83% (w/w).
19. Use of the strain of S. cerevisiae according to any one of items 1 to 10 for the production of yeast protein isolate.
20. Use of the strain of S. cerevisiae according to any one of items 1 to 10 for the production of a product comprising YPI, wherein the product is a dietary supplement, a nutritional supplement, a food supplement, a beverage supplement, a food additive or a feed additive.
21. Use of the soluble fraction obtained from the method according to item 13 as a food additive, e.g., as flavoring.

What is claimed:

1. A strain of Saccharomyces cerevisiae, obtainable from strain NFY-1816, deposited as CBS148506.

2. The strain of S. cerevisiae according to claim 1, wherein the strain is obtainable by breeding between a first strain and a second strain, wherein the first and/or the second strain is strain NFY-1816, deposited as CBS148506.

3. A strain of S. cerevisiae, wherein the strain is NFY-1816, deposited as CBS148506.

4. The strain of S. cerevisiae according to claim 1, comprising a protein content of at least about 50% (w/w), relative to the dry yeast biomass.

5. The strain of S. cerevisiae according to claim 1, capable of producing yeast protein in an amount of at least about 50 g/L of fermentation culture volume.

6. The strain of S. cerevisiae according to claim 1, wherein the strain enables a growth rate (μ) of at least about 0.29.

7. A method for producing yeast protein isolate, comprising culturing a strain of S. cerevisiae according to claim 1.

8. The method for producing yeast protein isolate according to claim 7, further comprising disruption of yeast cells to obtain a lysate, removal of cell wall components to obtain a cleared lysate, and isolation of the yeast protein from the lysate.

9. A yeast protein isolate obtainable by the method according to claim 7.

10. A yeast protein isolate produced by the method according to claim 8.

11. The yeast protein isolate according to claim 9, comprising a protein content of at least about 70% (w/w).

12. Use of the strain of S. cerevisiae according to claim 1 for the production of yeast protein isolate.

13. The strain of S. cerevisiae according to claim 3, comprising a protein content of at least about 50% (w/w), relative to the dry yeast biomass.

14. The strain of S. cerevisiae according to claim 3, capable of producing yeast protein in an amount of at least about 50 g/L of fermentation culture volume.

15. The strain of S. cerevisiae according to claim 3, wherein the strain enables a growth rate (μ) of at least about 0.29.

16. A method for producing yeast protein isolate, comprising culturing a strain of S. cerevisiae according to claim 3.

17. The method for producing yeast protein isolate according to claim 16, further comprising disruption of yeast cells to obtain a lysate, removal of cell wall components to obtain a cleared lysate, and isolation of the yeast protein from the lysate.

18. A yeast protein isolate obtainable by the method according to claim 16.

19. The yeast protein isolate according to claim 18, comprising a protein content of at least about 70% (w/w).

20. Use of the strain of S. cerevisiae according to claim 3 for the production of yeast protein isolate.
